(19) United States
(12) Patent Application Publication
(10) Pub. No.: US 2014/0228318 A1
(43) Pub. Date: Aug. 14, 2014

(54) Curcumin formulations and methods for making such formulations

(71) Applicants: Subhash Chauhan, Sioux Falls, SD (US); Meena Jaggi, Sioux Falls, SD (US); Murali Mohan Yallapu, Sioux Falls, SD (US)

(72) Inventors: Subhash Chauhan, Sioux Falls, SD (US); Meena Jaggi, Sioux Falls, SD (US); Murali Mohan Yallapu, Sioux Falls, SD (US)

(73) Assignee: Sanford Research/USD, Sioux Falls, SD (US)

(21) Appl. No.: 13/183,809

(22) Filed: Jul. 15, 2011

Related U.S. Application Data
(60) Provisional application No. 61/365,946, filed on Jul. 20, 2010.

(51) Int. Cl. A61K 47/48

(52) U.S. Cl. CPC A61K 47/48969
USPC 514/58; 536/103

(57) ABSTRACT

The present invention provides cyclodextrin-curcumin inclusion complexes, self-assemblies thereof; methods for making such inclusion complexes and self-assemblies, and methods for their use.

### CROSS REFERENCE

This application claims priority to U.S. Provisional Patent Application Ser. No. 61/365,946 filed Jul. 20, 2010, incorporated by reference herein in its entirety.

### STATEMENT OF U.S. GOVERNMENT RIGHTS

This work was supported in part by U.S. Department of Defense Grants PC073887 and PC073643. The U.S. government has certain rights in the invention.

### BACKGROUND

The incidence of prostate cancer is much higher in males in Western countries (≧60.6 cases per 100,000) compared to Asian countries (<5-10 cases per 100,000) [1, 2]. The current treatment for prostate cancer is a combination of surgery, radiation, and chemotherapy [3]. Clinically used therapeutic agents, such as mitoxantrone, estramustine, doxorubicin, etoposide, vinblastine, paclitaxel, carboplatin, vinorelbine, or combination drugs and anti-androgens, arrest cancer growth and reduce symptoms, which ultimately improves quality of life [4-8]. All these chemotherapeutic agents, however, have shown enormous toxicity to normal organs that leads to severe side effects [4-6]. In addition, most of the chemotherapeutic agents may not kill all prostate cancer cells and their repeated administration develops drug resistance or androgen refractory stage which is most difficult to cure [9]. Therefore, an urgent need exists to develop new classes or better drug formulations to treat prostate cancer which have fewer side effects to normal organs.

Curcumin (CUR), bis(4-hydroxy-3-methoxyphenyl)-1,6-diene-3,5-dione, is a low molecular weight polyphenol yellow compound derived from the rhizome of the plant Curcuma longa. Curcumin has a wide range of pharmacological applications such as anti-inflammation, anti-human immunodeficiency virus, anti-microbial, anti-oxidant, anti-parasitic, anti-mutagenic and anti-cancer [10-14] with low or no intrinsic toxicity. Curcumin is a well studied natural compound due to its putative cancer prevention and anti-cancer activities which are mediated through influencing multiple signaling pathways [15-20]. The CUR inhibitory effects on protein kinase C [21-23], epidermal growth factor receptor tyrosine kinase [24, 25] and cytotoxicity activities have been demonstrated in various human cancer cell lines [26]. In addition, CUR induces cell cycle arrest and/or apoptosis and blocks nuclear factor kappa B (NF-κB) activity which is an important cellular target of cancer cells [27]. Clinical trials of CUR at a oral dose of up to 12 g per day for 3 months report no toxicity issues [13, 28, 29]. Despite all these extraordinary anti-cancer properties, curcumin suffers from low solubility in aqueous solution (≈20 μg/mL) and undergoes rapid degradation at physiological pH [30] which results in low systemic bioavailability, poor pharmacokinetics, and greatly hampers its in vivo efficacy [31].

### SUMMARY OF THE INVENTION

In a first aspect, the present invention provides methods for preparing a curcumin formulation, comprising

(a) combining cyclodextrin dissolved in an aqueous solvent with curcumin in a non-aqueous solvent, wherein the combining occurs with agitation and under conditions to allow evaporation of the non-aqueous solvent; and

(b) separating solid and supernatant phases of the combination; wherein the supernatant phase contains cyclodextrin-curcumin inclusion complexes. In one preferred embodiment, the non-aqueous solvent is selected from the group consisting of dimethyl sulphoxide, dimethyl formamide, chloroform, dichloromethane, dioxane, ethanol, methanol, and acetone. In another preferred embodiment, the cyclodextrin is selected from the group consisting of β-cyclodextrin, α-cyclodextrin, γ-cyclodextrin, and combinations and modifications thereof. In a further preferred embodiment, the aqueous solvent is water.

In a second aspect, the present invention provides cyclodextrin-curcumin inclusion complexes, or self-assemblies thereof. In one embodiment, the cyclodextrin and curcumin are present in the inclusion complex, or self-assemblies thereof, in a ratio of between 20:1 and 1:1. In a further preferred embodiment, the cyclodextrin is selected from the group consisting of β-cyclodextrin, α-cyclodextrin, γ-cyclodextrin, and combinations and modifications thereof. In a further preferred embodiment, the cyclodextrin-curcumin inclusion complexes are made by the methods of the invention.

In a third aspect, the present invention provides a pharmaceutical dosage form comprising a cyclodextrin-curcumin self-assemblies of any embodiment of the invention.

### BRIEF DESCRIPTION OF THE FIGURES

FIG. 1. (A) Schematic illustration of curcumin supramolecular encapsulation (inclusion complexation) into β-cyclodextrin using solvent evaporation technique. (B) Solid powder samples of β-cyclodextrin (CD), curcumin (CUR) and CD-CUR inclusion complex (CD30). (C) Aqueous solubility of CD, CUR and CD-CUR inclusion complex (CD30) (5 mg/mL). (D) Curcumin loading capacity into different CD-CUR (CD5, CD10, CD20 and CD30) inclusion complexes.

FIG. 2. (A) Optical microscopic images of β-cyclodextrin (CD), curcumin (CUR) and β-cycicodextrin-curcumin (CD-CUR) inclusion complex films obtained from aqueous solutions. Original magnifications 200×. (B) Stability curves of different CD-CUR (CD5, CD10, CD20 and CD30) inclusion complexes with time.

FIG. 3. (A) FTIR spectra of curcumin (CUR), β-cyclodextrin (CD) and β-cyclodextrin-curcumin (CD-CUR) inclusion complex (CD30). Spectra of solid powders were recorded on ATR-FTIR place. (B) (a-c) ¹H-NMR spectra of β-cyclodextrin (CD), curcumin (CUR) and β-cyclodextrin-curcumin (CD-CUR) inclusion complex (CD30). Spectra were recorded for samples in d-DMSO solutions. (d) Chemical structures of CD and CUR are provided for the interpretation of the ¹H-NMR spectra.

FIG. 4. Scanning Electron Microscope (SEM) images of β-cyclodextrin (CD), curcumin (CUR), and β-cyclodextrin-curcumin (CD-CUR) inclusion complexes (CD5, CD10, CD20 and CD30). Scale bar on SEM image represents 400 μm.

FIG. 5. (A) Transmission Electron Microscopic (TEM) images of CD-CUR inclusion complexes (A) CD5, (B) CD10, (C) CD20 and (D) CD30. (E-F) Higher magnification images of CD30.

FIG. 6. (A) Putative schematic structures of cyclodextrin-curcumin (CD-CUR) inclusion complexes and (B) self-assembly or nano-assembly process of β-cyclodextrin (CD) and curcumin (CUR).

FIG. 7. (A) X-ray diffraction (XRD) patterns of β-cyclodextrin (CD), curcumin (CUR) and β-cyclodextrin-curcumin (CD-CUR) inclusion complex (CD30). (B) Differential Scanning calorimeter (DSC) endothermic curves of CD, CUR and CD-CUR inclusion complex (CD30). (C) Thermo-gravimetric curves of CD, CUR and CD-CUR inclusion complex (CD30).

FIG. 8. Cellular uptake of β-cyclodextrin, curcumin (CUR) or β-cyclodextrin-curcumin (CD-CUR) inclusion complex in prostate cancer cells. (A) Fluorescence images of C4-2 and DU145 prostate cancer cells treated with CD, CUR, or CD-CUR inclusion complex (CD30). Cells were treated with 10 μg of CD or CUR or equivalent CD-CUR inclusion complex for 6 hrs. After changing media, images were taken on Olympus BX 51 fluorescence microscope. Original Magnifications 200×. (B) Flow Cytometeric (FACS) analysis for cellular uptake of curcumin and different CD-CUR (CD5, CD10, CD20 and CD30) inclusion complex treated in DU145 prostate cancer cells. Mean fluorescence of cells treated with CUR or CD-CUR was measured by Accuri Flow Cytometer. Data represents average of 3 repeats. *p<0.05 represents significant difference from the curcumin uptake.

FIG. 9. β-cyclodextrin-curcumin (CD30) treatment suppresses cell proliferation in prostate cancer cells. (A and B) Prostate cancer cells (C4-2 and DU145) were treated with curcumin (CUR) or CD30 formulation for 48 hours. Cell proliferation was determined by MTS assay and normalized to cells treated with equivalent amounts of respective controls (DMSO for curcumin and cyclodextrin for CD30). Data represent mean±SE of 8 repeats of each treatment group. (C and D) Phase contrast microscopy images of 20 μM CUR or 20 μM CD30 treated prostate cancer cells. Note: CD30 has shown an improved therapeutic effect on prostate cancer cells compared to free CUR. Original Magnifications 200×.

FIG. 10. The effects of CD30 on clonogenic potential and apoptotic molecular event in prostate cancer cells. (A) CD30 inhibits the clonogenic potential of prostate cancer cells. C4-2 or DU145 cells (1000) were seeded in 6 well culture dishes and after 24 hrs treated with the indicated amounts of curcumin (CUR) or CD30. Cells were allowed to grow for 10 days; colonies were fixed and stained with hematoxylin. Images of colony forming assays were taken by digital camera. (B) Densities of formed colonies were measured by densitometer using AlphaEase Fc software and expressed as a percent of the DMSO or β-cyclodextrin (CD) control. Data represent mean of 3 repeats for each treatment (Mean±SE; *p<0.05, compared to the equivalent curcumin dose). (C) Immunoblot analysis for PARP cleavage in curcumin (CUR) or CD30 treated prostate cancer cells. β-actin was used as an internal loading control. Note: CD30 has shown enhanced PARP cleavage compared to curcumin.

FIG. 11. Standard plot of pure CUR in DMSO solution.

FIG. 12. Space-filling energy-minimized (MM2) molecular models showing different views of CD, CUR and CUR encapsulation in the CD cavities. The space-filling energy minimized molecular models are generated using Sporton'08 Demo Software (Wavefunctions, Inc., Irvine, Calif., USA).

### DETAILED DESCRIPTION OF THE INVENTION

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides methods for preparing a curcumin formulation, comprising

(a) combining cyclodextrin dissolved in an aqueous solvent with curcumin, or a pharmaceutically acceptable salt, ester, amide, or prodrug thereof, in a non-aqueous solvent, wherein the combining occurs with agitation and under conditions to allow evaporation of the non-aqueous solvent; and

(b) separating solid and supernatant phases of the combination; wherein the supernatant phase contains cyclodextrin-curcumin inclusion complexes.

As disclosed in detail herein, the methods of the invention can be used, for example, to prepare stable cyclodextrin-curcumin inclusion complexes to form self-assemblies comprising a plurality of cyclodextrin-curcumin inclusion complexes, which can be used to improve curcumin solubility, stability, bioavailability, and pharmacokinetic profile, thus greatly improving the pharmaceutical efficacy of curcumin in, for example, anti-inflammation, anti-human immunodeficiency virus, anti-microbial, anti-oxidant, anti-parasitic, anti-mutagenic and anti-cancer applications. The cyclodextrin-curcumin complexes are obtained in good yield, and are shown herein to provide increased stability of hydrophobic curcumin in aqueous media. The basis for improved solubility is shown herein to be the compatibility between cyclodextrin and curcumin in the solvent evaporation methods of the invention. The inclusion complexes and self-assemblies thereof provide for slow release of curcumin, thus providing further advantages.

Curcumin (CUR) is bis(4-hydroxy-3-methoxyphenyl)-1,6-diene-3,5-dione. As used herein, “curcumin” includes pharmaceutically acceptable salts, esters, amides, and prodrugs of curcumin, including but not limited to carboxylate salts, amino acid addition salts, esters, amides, and prodrugs of curcumin which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms. The term “salts” refers to the relatively non-toxic, inorganic and organic acid addition salts of curcumin. These salts can be prepared in situ during the curcumin isolation and purification or by separately reacting curcumin in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

Examples of pharmaceutically acceptable, non-toxic esters of curcumin include C₁-C₆ alkyl esters, wherein the alkyl group is a straight or branched, substituted or unsubstituted, C₅-C₇ cycloalkyl esters, as well as arylalkyl esters such as benzyl and triphenylmethyl. C₁-C₄ alkyl esters are preferred, such as methyl, ethyl, 2,2,2-trichloroethyl, and tert-butyl. Esters of the compounds of the present invention may be prepared according to conventional methods.

Examples of pharmaceutically acceptable, non-toxic amides of curcumin include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of curcumin may be prepared according to conventional methods.

The term “prodrug” refers to compounds that are rapidly transformed in vivo to yield curcumin, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

Any suitable amount of curcumin, or pharmaceutically acceptable salt, ester, amide, or prodrug thereof can be dissolved in the non-aqueous solvent as suitable for use in the methods of the invention. In one embodiment, the curcumin, or pharmaceutically acceptable salt, ester, amide, or prodrug thereof is dissolved in the non-aqueous solvent at a concentration of between 5-15 mg/ml. In various further embodiments, the curcumin, or pharmaceutically acceptable salt, ester, amide, or prodrug thereof is dissolved in the non-aqueous solvent at a concentration of between 6-14 mg/ml, 7-13 mg/ml; 7.5-12.5 mg/ml; 8-12 mg/ml; 9-11 mg/ml; or about 10 mg/ml.

Cyclodextrins are a family of compounds made up of sugar molecules bound together in a ring (cyclic oligosaccharides). Cyclodextrins are composed of 5 or more α-D-glucopyranoside units linked 1->4. Typical cyclodextrins contain a number of glucose monomers ranging from six to eight units in a ring, creating a cone shape. Any suitable cyclodextrin may be used. In one embodiment, the cyclodextrin is selected from the group consisting of β-cyclodextrin (seven sugar ring molecule), α-cyclodextrin (six membered sugar ring molecule), γ-cyclodextrin (eight sugar ring molecule), and modifications thereof.

Any suitable concentration of cyclodextrin that can be dissolved in the aqueous solvent is suitable for the methods of the invention. In one embodiment, the cyclodextrin is dissolved in the aqueous solvent at a concentration of between 4-12 mg/ml. In further embodiments, the cyclodextrin is dissolved in the aqueous solvent at a concentration of between 5-11 mg/mg; 6-10 mg/ml; 7-9 mg/ml, or about 8 mg/ml. Any suitable non-aqueous solvent compatible with curcumin may be used. In one embodiment, the non-aqueous solvent is selected from the group consisting of dimethyl sulphoxide, dimethyl formamide, chloroform, dichloromethane, dioxane, ethanol, methanol, and acetone.

Any suitable aqueous solvent in which cyclodextrins can be dissolved may be used. In various embodiments, the aqueous solvent is water, NaOH solutions, or dimethylsulfoxide.

The combining can be carried out under any suitable conditions involving agitation and under conditions to allow evaporation of the non-aqueous solvent, which can be determined by those of skill in the art based on the teachings herein. In one embodiment, the combining is carried out at room temperature to 25° C.

In one embodiment, the combining comprises stirring at 200-600 rpm, and wherein the combination is stirred in a container with no cap, for at least part of the reaction (1 hour, 2 hours, 3 hours, 4 hours, etc.) to facilitate evaporation of the non-aqueous solvent. Such stirring may be carried out for any suitable length of time, which can be determined by those of skill in the art based on the teachings herein. In one embodiment, stirring is carried out for between 1-24 hours.

Any suitable ratio of cyclodextrin and curcumin, or pharmaceutically acceptable salt, ester, amide, or prodrug thereof can be used in the combining step, as is appropriate for a given purpose. In one embodiment, the combining comprises combining cyclodextrin (CD) (such as β-cyclodextrin) and curcumin, or pharmaceutically acceptable salt, ester, amide, or prodrug thereof (CUR), in a molar ratio of between 20:1 and 1:1; in various further embodiments, in a CD:CUR molar ratio of between 19:1 and 1:1; 18:1 and 1:1, 17:1 and 1:1, 16:1 and 1:1; 15:1 and 1:1; 14:1 and 1:1; 13:1 and 1:1; 12:1 and 1:1; 11:1 and 1:1; 10:1 and 1:1; 9:1 and 1:1; 8:1 and 1:1; 7:1 and 1:1; 6:1 and 1:1; 5:1 and 1:1; 4:1 and 1:1; 3:1 and 1:1; 2:1 and 1:1 19:1 and 2:1; 18:1 and 2:1, 17:1 and 2:1, 16:1 and 2:1; 15:1 and 2:1; 14:1 and 2:1; 13:1 and 2:1; 12:1 and 2:1; 11:1 and 2:1; 10:1 and 2:1; 9:1 and 2:1; 8:1 and 2:1; 7:1 and 2:1; 6:1 and 2:1; 5:1 and 2:1; 4:1 and 2:1; 3:1 and 2:1; 19:1 and 3:1; 18:1 and 3:1, 17:1 and 3:1, 16:1 and 3:1; 15:1 and 3:1; 14:1 and 3:1; 13:1 and 3:1; and 12:1 and 3:1; 11:1 and 3:1; 10:1 and 3:1; 9:1 and 3:1; 8:1 and 3:1; 7:1 and 3:1; 6:1 and 3:1; 5:1 and 3:1; 4:1 and 3:1; 19:1 and 4:1; 18:1 and 4:1, 17:1 and 4:1, 16:1 and 4:1; 15:1 and 4:1; 14:1 and 4:1; 13:1 and 4:1; and 12:1 and 4:1; 11:1 and 4:1; 10:1 and 4:1; 9:1 and 4:1; 8:1 and 4:1; 7:1 and 4:1; 6:1 and 4:1; 5:1 and 4:1; 19:1 and 5:1; 18:1 and 5:1, 17:1 and 5:1, 16:1 and 5:1; 15:1 and 5:1; 14:1 and 5:1; 13:1 and 5:1; and 12:1 and 5:1; 11:1 and 5:1; 10:1 and 5:1; 9:1 and 5:1; 8:1 and 5:1; 7:1 and 5:1; 6:1 and 5:1; 19:1 and 6:1; 18:1 and 6:1, 17:1 and 6:1, 16:1 and 6:1; 15:1 and 6:1; 14:1 and 6:1; 13:1 and 6:1; and 12:1 and 6:1; 11:1 and 6:1; 10:1 and 6:1; 9:1 and 6:1; 8:1 and 6:1; 7:1 and 6:1; 19:1 and 7:1; 18:1 and 7:1, 17:1 and 7:1, 16:1 and 7:1; 15:1 and 7:1; 14:1 and 7:1; 13:1 and 7:1; and 12:1 and 7:1; 11:1 and 7:1; 10:1 and 7:1; 9:1 and 7:1; 8:1 and 7:1; 19:1 and 8:1; 18:1 and 8:1, 17:1 and 8:1, 16:1 and 8:1; 15:1 and 8:1; 14:1 and 8:1; 13:1 and 8:1; and 12:1 and 8:1; 11:1 and 8:1; 10:1 and 8:1; 9:1 and 8:1; 19:1 and 9:1; 18:1 and 9:1, 17:1 and 9:1, 16:1 and 9:1; 15:1 and 9:1; 14:1 and 9:1; 13:1 and 9:1; and 12:1 and 9:1; 11:1 and 9:1; 10:1 and 9:1; 19:1 and 10:1; 18:1 and 10:1, 17:1 and 10:1, 16:1 and 10:1; 15:1 and 10:1; 14:1 and 10:1; 13:1 and 10:1; and 12:1 and 10:1; 11:1 and 10:1; 19:1 and 11:1; 18:1 and 11:1, 17:1 and 11:1, 16:1 and 11:1; 15:1 and 11:1; 14:1 and 11:1; 13:1 and 11:1; and 12:1 and 11:1; 19:1 and 12:1; 18:1 and 12:1, 17:1 and 12:1, 16:1 and 12:1; 15:1 and 12:1; 14:1 and 12:1; 13:1 and 12:1; 19:1 and 13:1; 18:1 and 13:1, 17:1 and 13:1, 16:1 and 13:1; 15:1 and 13:1; 14:1 and 13:1; 19:1 and 14:1; 18:1 and 14:1, 17:1 and 14:1, 16:1 and 14:1; 15:1 and 14:1; 19:1 and 15:1; 18:1 and 15:1, 17:1 and 15:1, 16:1 and 15:1; 19:1 and 16:1; 18:1 and 16:1, 17:1 and 16:1; 19:1 and 17:1; 18:1 and 17:1; or 19:1 and 18:1.

Any suitable starting total weight of cyclodextrin and curcumin can be employed in the methods of the invention to achieve a particular inclusion complex size and amount of curcumin loaded therein. As noted in the examples that follow, complexes of up to 26% curcumin can be produced using the methods of the invention.

Any suitable means for separating solid and supernatant phases of the combination can be used. In one embodiment, the separating comprises centrifugation. Any suitable centrifugation conditions may be used. In one embodiment, the separating step comprises centrifugation at between 800-1200 rpm for a suitable time period, which will depend at least in part on the volume of the combination, and which can be determined by those of skill in the art based on the teachings herein.

In a further embodiment, the methods further comprise isolation of cyclodextrin-curcumin inclusion complexes from the supernatant. Any suitable means for isolation of the inclusion complex can be used. In various embodiments, the isolation comprises freeze-drying or spray-drying the supernatant. Suitable conditions for freeze-drying can be determined by those of skill in the art based on the teachings herein.

Thus, the present invention provides isolated cyclodextrin-curcumin inclusion complexes prepared by the method of any embodiment, or combination of embodiments of the methods for making the complexes disclosed herein.

In a second aspect, the present invention provides cyclodextrin-curcumin inclusion complexes, or self-assemblies thereof formed by a complexation reaction of a plurality of the inclusion complexes. Any embodiments, or combinations of embodiments, of cyclodextrin and curcumin as discussed herein, are also suitable embodiments in this aspect of the invention. Thus, for example, in one embodiment, the cyclodextrin and curcumin, or pharmaceutically acceptable salt, ester, amide, or prodrug thereof, are present in the inclusion complex in a ratio of between 20:1 and 1:1; in various further embodiments, in a ratio of between 19:1 and 1:1; 18:1 and 1:1, 17:1 and 1:1, 16:1 and 1:1; 15:1 and 1:1; 14:1 and 1:1; 13:1 and 1:1; 12:1 and 1:1; 11:1 and 1:1; 10:1 and 1:1; 9:1 and 1:1; 8:1 and 1:1; 7:1 and 1:1; 6:1 and 1:1; 5:1 and 1:1; 4:1 and 1:1; 3:1 and 1:1; 2:1 and 1:1 19:1 and 2:1; 18:1 and 2:1, 17:1 and 2:1, 16:1 and 2:1; 15:1 and 2:1; 14:1 and 2:1; 13:1 and 2:1; 12:1 and 2:1; 11:1 and 2:1; 10:1 and 2:1; 9:1 and 2:1; 8:1 and 2:1; 7:1 and 2:1; 6:1 and 2:1; 5:1 and 2:1; 4:1 and 2:1; 3:1 and 2:1; 19:1 and 3:1; 18:1 and 3:1, 17:1 and 3:1, 16:1 and 3:1; 15:1 and 3:1; 14:1 and 3:1; 13:1 and 3:1; and 12:1 and 3:1; 11:1 and 3:1; 10:1 and 3:1; 9:1 and 3:1; 8:1 and 3:1; 7:1 and 3:1; 6:1 and 3:1; 5:1 and 3:1; 4:1 and 3:1; 19:1 and 4:1; 18:1 and 4:1, 17:1 and 4:1, 16:1 and 4:1; 15:1 and 4:1; 14:1 and 4:1; 13:1 and 4:1; and 12:1 and 4:1; 11:1 and 4:1; 10:1 and 4:1; 9:1 and 4:1; 8:1 and 4:1; 7:1 and 4:1; 6:1 and 4:1; 5:1 and 4:1; 19:1 and 5:1; 18:1 and 5:1, 17:1 and 5:1, 16:1 and 5:1; 15:1 and 5:1; 14:1 and 5:1; 13:1 and 5:1; and 12:1 and 5:1; 11:1 and 5:1; 10:1 and 5:1; 9:1 and 5:1; 8:1 and 5:1; 7:1 and 5:1; 6:1 and 5:1; 19:1 and 6:1; 18:1 and 6:1, 17:1 and 6:1, 16:1 and 6:1; 15:1 and 6:1; 14:1 and 6:1; 13:1 and 6:1; and 12:1 and 6:1; 11:1 and 6:1; 10:1 and 6:1; 9:1 and 6:1; 8:1 and 6:1; 7:1 and 6:1; 19:1 and 7:1; 18:1 and 7:1, 17:1 and 7:1, 16:1 and 7:1; 15:1 and 7:1; 14:1 and 7:1; 13:1 and 7:1; and 12:1 and 7:1; 11:1 and 7:1; 10:1 and 7:1; 9:1 and 7:1; 8:1 and 7:1; 19:1 and 8:1; 18:1 and 8:1, 17:1 and 8:1, 16:1 and 8:1; 15:1 and 8:1; 14:1 and 8:1; 13:1 and 8:1; and 12:1 and 8:1; 11:1 and 8:1; 10:1 and 8:1; 9:1 and 8:1; 19:1 and 9:1; 18:1 and 9:1, 17:1 and 9:1, 16:1 and 9:1; 15:1 and 9:1; 14:1 and 9:1; 13:1 and 9:1; and 12:1 and 9:1; 11:1 and 9:1; 10:1 and 9:1; 19:1 and 10:1; 18:1 and 10:1, 17:1 and 10:1, 16:1 and 10:1; 15:1 and 10:1; 14:1 and 10:1; 13:1 and 10:1; and 12:1 and 10:1; 11:1 and 10:1; 19:1 and 11:1; 18:1 and 11:1, 17:1 and 11:1, 16:1 and 11:1; 15:1 and 11:1; 14:1 and 11:1; 13:1 and 11:1; and 12:1 and 11:1; 19:1 and 12:1; 18:1 and 12:1, 17:1 and 12:1, 16:1 and 12:1; 15:1 and 12:1; 14:1 and 12:1; 13:1 and 12:1; 19:1 and 13:1; 18:1 and 13:1, 17:1 and 13:1, 16:1 and 13:1; 15:1 and 13:1; 14:1 and 13:1; 19:1 and 14:1; 18:1 and 14:1, 17:1 and 14:1, 16:1 and 14:1; 15:1 and 14:1; 19:1 and 15:1; 18:1 and 15:1, 17:1 and 15:1, 16:1 and 15:1; 19:1 and 16:1; 18:1 and 16:1, 17:1 and 16:1; 19:1 and 17:1; 18:1 and 17:1; or 19:1 and 18:1. In a further embodiment, the cyclodextrin in the isolated cyclodextrin-curcumin inclusion complex comprises β-cyclodextrin.

In a further embodiment, the cyclodextrin-curcumin inclusion complexes, or self-assemblies thereof, comprise between 1% and 26% curcumin. In further embodiments, the inclusion complexes comprise between 2%-26%, 3%-26%, 4%-26%, 5%-26%, 6%-26%, 7%-26%, 8%-26%, 9%-26%, 10%-26%, 11-%-26%, 12%-26%, 13%-26%, 14%-26%, 15-%-26%, 16-%-26%, 17-%-26%, 18%-26%, 19%-26%, 20%-26%, 21%-26%, 22%-26%, 23%-26%, 24%-26%, 25%-26%, or 26% curcumin. The total weight of cyclodextrin-curcumin inclusion complexes, or self-assemblies thereof, can be any suitable weight for a given purpose.

The diameter of individual inclusion complexes or self-assemblies thereof can be any suitable size for a given purpose. In one embodiment, the diameter of self-assemblies range from 50-500 nm; in various further embodiments, the diameter ranges between 60-500 nm; 70-500 nm; 80-500 nm; 90-500 nm; 100-500 nm; 50-450 nm; 60-450 nm; 70-450 nm; 80-450 nm; 90-450 nm; 100-450 nm; 50-400 nm; 60-400 nm; 70-400 nm; 80-400 nm; 90-400 nm; 100-400 nm; 50-350 nm; 60-350 nm; 70-350 nm; 80-350 nm; 90-350 nm; 100-350 nm; 50-300 nm; 60-300 nm; 70-300 nm; 80-300 nm; 90-300 nm; 100-300 nm; 50-250 nm; 60-250 nm; 70-250 nm; 80-250 nm; 90-250 nm; or 100-250 nm.

In a third aspect, the present invention provides a pharmaceutical dosage form comprising the cyclodextrin-curcumin self-assemblies of any embodiment or combination of embodiments of the invention. As will be understood by those of skill in the art based on the teachings herein, the dosage forms may contain self-assemblies with combinations of different sizes, percent of curcumin, etc.

In another embodiment, the cyclodextrin in the cyclodextrin-curcumin self-assemblies comprises β-cyclodextrin. In another embodiment, the cyclodextrin and curcumin are present in the self-assemblies in a ratio of between 20:1 and 1:1, or any other embodiments or combinations of embodiments as disclosed above.

Any suitable dosage form may be used for delivery of the pharmaceutical compositions of the invention, as may be suitable for any given use of the pharmaceutical composition. In non-limiting embodiments, the dosage form is formulated into a form selected from the group consisting of tablets, gelcaps, softgels, and capsules.

The self-assemblies of the invention can be administered as the sole active pharmaceutical agent, or they can be used in combination with one or more other compounds useful for carrying out the methods of the invention. When administered as a combination, the other therapeutic agents can be formulated as separate compositions that are given at the same time or different times, or the other therapeutic agents can be given as a single composition with the self-assemblies.

The self-assemblies may be made up in a solid form (including granules, powders or suppositories) or in a liquid form (e.g., solutions, suspensions, or emulsions). The self-assemblies of the invention may be applied in a variety of solutions and may be subjected to conventional pharmaceutical operations such as sterilization and/or may contain conventional adjuvants, such as preservatives, stabilizers, wetting agents, emulsifiers, buffers etc.

The self-assemblies of the invention may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of the invention and a pharmaceutically acceptable carrier. The pharmaceutical compositions containing self-assemblies of the invention may be in a form suitable for oral use, for example, as tablets, troches, lozenges, aqueous or oily suspensions, dispersible powders or granules, emulsion, hard or soft capsules, or syrups or elixirs.

Self-assemblies intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the self-assemblies in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the self-assemblies are mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

In a fourth aspect, the present invention provides methods for treating one or more disorder selected from the group consisting of inflammation, infection, stroke, and cancer, comprising administering to a subject in need thereof a cyclodextrin-curcumin inclusion complex, or self-assemblies thereof according to any embodiment or combination of embodiments herein, in an amount effective to treat the inflammation, infection, stroke, or cancer.

The subject can be any suitable subject suffering from inflammation, infection, or cancer. In one embodiment, the subject is a mammal, such as a human. In one embodiment, the infection is a viral (including but not limited to human immunodeficiency virus), bacterial, or parasitic infection. Any suitable cancers may be treated, including but not limited to prostate cancer, pancreatic cancer, colon cancer, ovarian cancer, and cervical cancer.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

Amounts effective for these uses depend on factors including, but not limited to, the nature of the compound (specific activity, etc.), the route of administration, the stage and severity of the disorder, the weight and general state of health of the subject, and the judgment of the prescribing physician. It will be understood that the amount of the cyclodextrin-curcumin inclusion complex or self-assembly thereof actually administered will be determined by a physician, in the light of the above relevant circumstances. In one non-limiting embodiment, an amount effective is an amount that provides between 10 mg and 10 g of curcumin per day.

### EXAMPLES

Curcumin, a hydrophobic polyphenolic compound derived from the rhizome of the herb Curcuma longa, possesses a wide range of biological applications including cancer therapy. However, its prominent application in cancer treatment is limited due to sub-optimal pharmacokinetics and poor bioavailability at the tumor site. In order to improve its hydrophilic and drug delivery characteristics, we have developed a β-cyclodextrin (CD) mediated curcumin drug delivery system via encapsulation technique. Curcumin encapsulation into the CD cavity was achieved by inclusion complex mechanism. Curcumin encapsulation efficiency was improved by increasing the ratio of curcumin to CD. The formations of CD-curcumin complexes were characterized by Fourier Transform Infra-red (FTIR), Differential Scanning calorimetry (DSC), Thermo-gravimetric Analysis (TGA), Scanning Electron Microscope (SEM), and Transmission Electron Microscope (TEM) analyses. An optimized CD-curcumin complex (CD30) was evaluated for intracellular uptake and anti-cancer activity. Cell proliferation and clonogenic assays demonstrated that β-cyclodextrin-curcumin self-assembly enhanced curcumin delivery and improved its therapeutic efficacy in prostate cancer cells compared to free curcumin.

In order to explore the cyclodextrin carrier properties for delivery of curcumin, we prepared a self-assembly of β-cyclodextrin and curcumin via an inclusion complex mechanism [43] in which the cyclomaltoheptaose structure acts as a drug shuttle while hydroxyl groups of cyclodextrin import good solubility to the system. The developed self-assemblies, i.e., β-cyclodextrin-curcumin (CD-CUR) inclusion complexes, were confirmed by spectroscopy (FTIR, ¹H-NMR), thermal studies (DSC and TGA), X-ray diffraction (XRD) and microscopic studies (SEM and TEM). An improved intracellular uptake of CD-CURs by cancer cells was observed. The anti-cancer efficacy of these formulations was evaluated by cell proliferation and clonogenic assays.

### 2. Materials and Methods

### 2.1. Materials

β-cyclodextrin, curcumin (≧95% purity, (E,E)-1,7-bis(4-Hydroxy-3-methoxyphenyl)-1,6-heptadiene-3,5-dione), acetone (≧99.5, ACS reagent grade), dimethylsulfoxide (DMSO) (ACS reagent, UV spectrophotometry grade, ≧99.9%) were purchased from Sigma Chemical Co. (St Louis, Mo., USA).

### 2.2. Preparation of Inclusion Complexes

β-cyclodextrin (CD) (40 mg) was dissolved in 8 mL deionized (DI) water in a 20 mL glass vial (Fisher Scientific, Pittsburgh, Pa., USA) containing a magnetic bar. To this solution, varying amounts of curcumin [2 mg (5%), 4 mg (10%), 8 mg (20%) and 12 mg (30%)] in 500 μL acetone were made while stirring at 400 rpm. Stirring was allowed without a cap to evaporate the acetone. The solution was stirred overnight, centrifuged at 1000 rpm for 5 min, and a supernatant containing highly water soluble β-cyclodextrin-curcumin (CD-CUR) inclusion complexes was recovered by freeze drying (Labconco Freeze Dry System, Labconco, Kansas City, Mo., USA; −48° C., 133×10⁻³ mBar). The CD-CUR inclusion complexes were stored at 4° C. until further use. The resultant CD-CUR inclusion complexes were designated as CD5, CD10, CD20 and CD30 based on the % of CUR employed in the preparations. The yield of CD5, CD10, CD20 and CD30 inclusion complexes were found to be 34.7, 36.4, 38.5, and 38.2 mg.

### 2.3. Determination of Curcumin (CUR) Loading

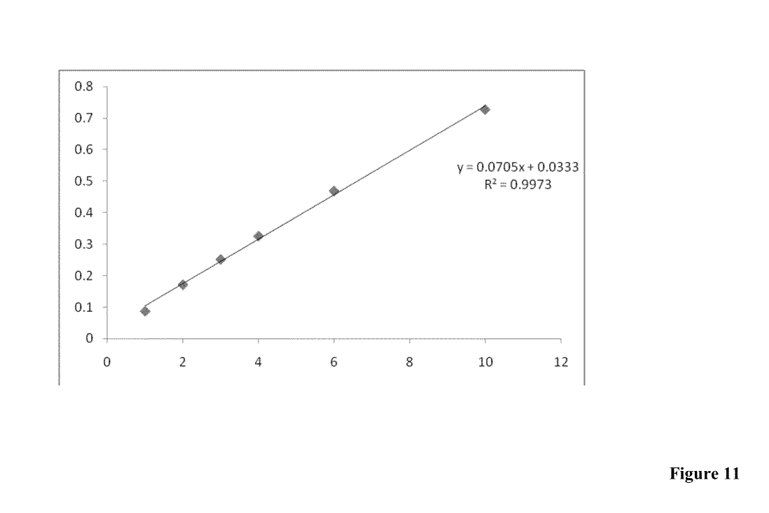

CD-CUR (1 mg) inclusion complex was dissolved in 50 mL dimethylsulfoxide (DMSO) to extract CUR in DMSO for the loading estimations. The CD-CUR inclusion complex sample in DMSO was gently shaken on a shaker (Labnet S 2030-RC, RPM 150, Labnet International, Woodbridge, N.J.) for 24 hrs at room temperature in the dark. The CUR extracted DMSO solution was centrifuged at 14,000 rpm (Centrifuge 5415D, Eppendorf AG, Hamburg, Germany) to remove clumps of CD and a clear supernatant DMSO solution containing CUR was collected and used for the estimations. The CUR concentration was determined by a standard UV-Vis spectrophotometer method (Biomate 3, Thermo Electron Corporation, Hudson, N.H., USA) at 450 nm [32]. A standard plot of CUR in DMSO (0-10 μg/mL) was prepared under identical conditions. A detailed loading calculation of CUR in CD-CUR complex (CD5) is as follows: Standard CUR solutions in DMSO (1-10 μg/mL) was prepared and measured on UV-Vis spectrophotometer at 450 nm. The absorbencies of the solutions are tabulated in Table 1. A standard plot was made from these absorbance values (FIG. 11).

TABLE 1
Absorbance values for 1-10 μg/mL CUR
containing DMSO solutions
CUR Standard solutions

| μg/mL | UV-vis absorbance (a.u.) |
|---|---|
| 1 | 0.088 |
| 2 | 0.172 |
| 3 | 0.252 |
| 4 | 0.326 |
| 6 | 0.469 |
| 10 | 0.727 |

1 mg of CD 5 inclusion complex was dissolved in 5 mL DMSO (Solution A). CUR was extracted in DMSO and absorbency was measured at 450 nm using UV-Vis spectrophotometer, i.e., 0.98.

$$CUR in Solution A = ( Absorbance value - intercept of standard plot ) / ( slope of standard plot ) = ( 0.98 - 0.0333 ) / ( 0.0705 ) = 3.0028 μg \text{/} mL$$

$$CUR containing in 1 mg CD - CUR complex = 3.0028 \times 5 = 61.75177 μg$$

$$CUR in 1 mg of CD 5 inclusion complex ( CUR Loading ) = 61.75177 μg$$

### 2.4. Compatibility of CD-CUR Inclusion Complexes

To determine compatibility of CD-CUR inclusion complexes, an optical microscopy study was performed. For this study, CD5, CD10, CD20 and CD30 of CD-CUR inclusion complexes (500 μg/mL), and equivalent concentrations of CUR or β-cyclodextrin aqueous solutions were prepared. Two drops of these solutions were placed on a glass slide and allowed to dry under fume hood overnight. Care was taken to protect from light and not deposit any dust on glass slides. These cover slides containing samples were analyzed using optical microscopy (Olympus BX 41 microscope; Olympus, Center Valley, Pa., USA). All the images were taken at 200× magnification.

### 2.5. In Vitro Stability of CD-CUR Inclusion Complexes

Curcumin (10 mg) containing CD-CUR inclusion complexes (CD5, CD10, CD20 and CD30) were separately dispersed in 5 mL of physiological buffer (1×PBS, 0.01 M PBS, pH 7.4). These suspensions were incubated at 37° C. under gentle shaking on a shaker (Labnet S 2030-RC, RPM 150, Labnet International, Woodbridge, N.J.). Curcumin retention in CD-CUR inclusion complexes was determined at different time intervals. The % curcumin retention was calculated using the following equation: % CUR retention=[(CUR in inclusion complex−Released curcumin)/(CUR inclusion complex)]×100.

### 2.6. Characterization of CD-CUR Inclusion Complexes

Fourier Transform Infrared (FTIR) spectroscopy: FTIR spectra of CD, CUR and CD-CUR inclusion complexes were performed using a Smiths Detection IlluminatIR FT-IR microscope (Danbury, Conn., USA) with diamond ATR objective. FTIR spectra of samples were acquired by placing fluffy powder on the tip of the ATR. Data was acquired between 4000-750 cm⁻¹ at a scanning speed of 4 cm⁻¹ and 32 scans. The average of 32 scans data was presented as FTIR spectra.

¹H-NMR spectroscopy: All spectra were recorded in DMSO-d₆ using Bruker Avance DRX 500 MHz NMR spectrophotometer (Bruker BioSpin Corp., Billerica, Mass., USA). The following parameters were used during the NMR experiments: number of scans, 64; relaxation delay, 1.0 s; and pulse degree, 25° C. The chemical shifts are presented in terms of parts per million (ppm) with tetramethylsilane (TMS) as the internal reference.

Differential Scanning Calorimetry (DSC): DSC analysis of CD, CUR and CD-CUR inclusion complexes were performed using TA Instruments Q200 Differential Scanning calorimeter (TA Instruments, New Castle, Del., USA) at The Applied Polymer Research Center, The University of Akron (Project #139-10APRC). The cell constant calibration method was employed to study the DSC patterns of the samples from 25° C. to 300° C. at a heating ramp of 10° C., under a constant flow (100 mL/min) of nitrogen gas.

Thermo-gravimetric Analysis (TGA): The thermal history of CD, CUR and CD-CUR inclusion complexes were evaluated on a TA Instruments Q50 Thermogravimetric Analyzer (TA Instruments, New Castle, Del., USA) at The Applied Polymer Research Center, The University of Akron (Project #139-10APRC) from 25° C. to 700° C. at a heating ramp of 10° C., under a constant flow (100 mL/min) of nitrogen gas. This study followed 20STD800 standard rubber analysis method.

X-ray Diffraction: X-ray diffraction measurements of CD, CUR and CD-CUR inclusion complexes were recorded using a D/Max-B Rikagu diffractometer (Rigaku Americas Corp, Woodlands, Tex.) using Cu radiation at λ=0.1546 nm, running at 40 kV and 40 mA. For this study, samples were mounted on double sided silicone tape and measurements were performed from 20° to 80° at a scan speed of 2° per minute.

Scanning Electron Microscopy (SEM): The surface morphology of CD, CUR and CD-CUR inclusion complexes was studied using a Quanta 450 Scanning Electron Microscope (S, No. D9234, FEI™, Hillsboro, Oreg.) at an accelerating voltage of 5 kV. Dry samples were spread on double sided carbon tape, mounted on SEM stage before scanning.

Transmission Electron Microscopy (TEM): Transmission Electron Microscopy was employed to view the exact morphology of samples suspended in water. To prepare TEM samples, 2-3 drops of aqueous solution of CD-CUR inclusion complexes were placed on a 200 mesh formvar-coated copper TEM grid (grid size: 97 μm) (Ted Pella, Inc., Redding, Calif., USA). Excess solution was removed using a piece of fine filter paper and the samples were allowed to air dry overnight prior to imaging particles. The morphology of samples was observed under JEOL-1210 transmission electron microscope (JEOL, Tokyo, Japan) operating at 60 kV.

### 2.7. Cell Culture

Prostate cancer cell lines C4-2 and DU145 were generously provided by Dr. Meena Jaggi. These cells were maintained as monolayer cultures (C4-2) in RPMI-1640 medium or in MEM medium (DU145) (HyClone Laboratories, Inc., Logan, Utah) and supplemented with 10% fetal bovine serum (Atlanta Biologicals, Lawrenceville, Ga.) and 1% penicillin-streptomycin (Gibco BRL, Grand Island, N.Y.) at 37° C. in a humidified atmosphere (5% CO₂).

### 2.8. CUR and CD-CUR Inclusion Complexes Cellular Uptake

For a comparative visualization of CUR and CD-CUR (CD30) inclusion complexes uptake in prostate (C4-2 and DU145) cancer cells, 5×10⁵ cells were seeded in 6-well plates in 2 mL medium. After cells were attached to the plate, media was replaced with 10 μg free curcumin or equivalent curcumin containing CD-CUR inclusion complexes. After 6 hrs, cells were examined under an Olympus BX 51 fluorescence microscope (Olympus, Center Valley, Pa.).

To investigate which CD-CUR inclusion complex (CD5, CD10, CD20, CD30) has superior CUR uptake, DU145 prostate cancer cells (5×10⁵) were seeded in 6-well plates (2 mL medium), and allowed to attach overnight. Cells were then treated with 10 μg CUR in each well or equivalent CD-CUR inclusion complexes. After 1 or 2 days, cells were washed twice with 1×PBS, trypsinized, centrifuged and collected in 2 mL media. The cell suspension (50 μl) was injected into an Acuri C6 flow cytometer (Accuri Cytometers, Inc., Ann Arbor, Mich., USA) to determine the fluorescence levels in the FL1 channel (488 excitation, Blue laser, 530±15 nm, FITC/GFP).

### 2.9. In Vitro Cytotoxicity (MTS Assay)

To examine the anti-cancer activity of the CD-CUR inclusion complex (CD-30), cell proliferation assays were performed using C4-2 and DU145 prostate cancer cell lines. Prostate cancer cells (5000 cells/well in 100 μL media) were seeded in 96-well plates and allowed to attach overnight. The next day, media was replaced with different concentrations (5-40 μM) of CUR or CD30. Media containing equivalent amounts of CD in PBS or DMSO was used as the control. After day 2, the media was replaced with fresh media and the anti-proliferative effect of the CUR and CD30 was determined using a standard 3-(4,5-dimethylthiazol-2-yl)-2,5-diphenyltetrazolium bromide (MTS) based colorimetric assay (CellTiter 96 AQₑₒᵤₛ, Promega, Madison, Wis.). The reagent (25 μL/well) was added to each well and plates were incubated for 3 hrs at 37° C. The color intensity was measured at 492 nm using a microplate reader (BioMate 3 UV-Vis spectrophotometer, Thermo Electron Corporation, Hudson, N.H.). The anti-proliferative effect of CUR and CD30 treatments was calculated as a percentage of cell growth with respect to the DMSO and CD in PBS controls. Just before adding MTS reagents, representative phase contrast microscope images of cells were taken using an Olympus BX 41 microscope (Olympus, Center Valley, Pa., USA).

### 2.10. Colony Formation Assay

Prostate cancer cells (1000) were seeded in 2 mL media in 6-well plates and allowed 2-3 days to attach and initiate the colony formations. Then cells were treated with different concentrations (2-10 μM) of CUR or CD30 over a period of 10 days. The plates were then washed twice with PBS, fixed in chilled methanol, stained with hematoxylin (Fisher Scientific, Fair Lawn, N.J., USA), washed with water and air dried. The number of colonies was counted by a MultiImage™ Cabinet (Alpha Innotech Corporation, San Leandro, Calif., USA) using AlphaEase Fc software. The percent of colonies was calculated using the number of colonies formed in treatment divided by number of colonies formed in DMSO or CD in PBS controls.

### 2.11. Immunoblot Assay

To investigate the possible mechanisms that are involved in the process of apoptosis or cell death, cells were collected after the treatment with CUR and CD30 and processed for protein extraction and Western blotting. In brief, C4-2 and DU145 cancer cells were grown overnight and treated with 20 μM curcumin or 20 μM CD30 for 48 hrs. The cells were then washed with PBS and scraped in SDS buffer (Santa Cruz Biotechnology, Santa Cruz, Calif., USA) and kept at 4° C. for 30 minutes. The cell lysates were passed through one freeze-thaw cycle and tip sonicated on ice bath for 30 sec (Sonic Dismembrator Model 100, Fisher Scientific). The protein concentrations of cell lysates were determined using SYPRO Orange (Invitrogen, Carlsbad, Calif., USA) method. The cell lysates were heated at 90° C. for 5 min, cooled down to 4° C., centrifuged at 14,000 rpm for 3 min and supernatants were collected. The proteins were resolved by SDS-PAGE (4-20%) gel electrophoresis and transferred onto (Polyvinylidene difluoride) (PVDF) membrane. The membranes were blocked in 5% nonfat dry milk in TBS (Tris buffered saline) for 1 hr and incubated overnight at 4° C. with primary antibody specific to β-actin/PARP. The membranes were washed (4×10 min) in TBS-T at room temperature and then probed with 1:2000 diluted horseradish peroxidase-conjugated goat anti-mouse or goat anti-rabbit secondary antibodies (Promega, Madison Wis., USA) for 1 hr at room temperature and washed (5×10 min) with TBS-T. The signal was detected with the Lumi-Light detection kit (Roche, Nutley, N.J.) and a BioRad Gel Doc (BioRad, Hercules, Calif.).

### 2.12. Statistical Analysis

All results were processed using Microsoft Excel 2007 software and expressed as mean±standard error of the mean (S.E.M.). Statistical analyses were performed using an unpaired, two tailed student t-test. A value of p<0.05 was considered significant. All graphs were plotted using Origin 6.1 software.

### 3. Results and Discussion

The main focus of the present study was to develop a β-cyclodextrin-curcumin

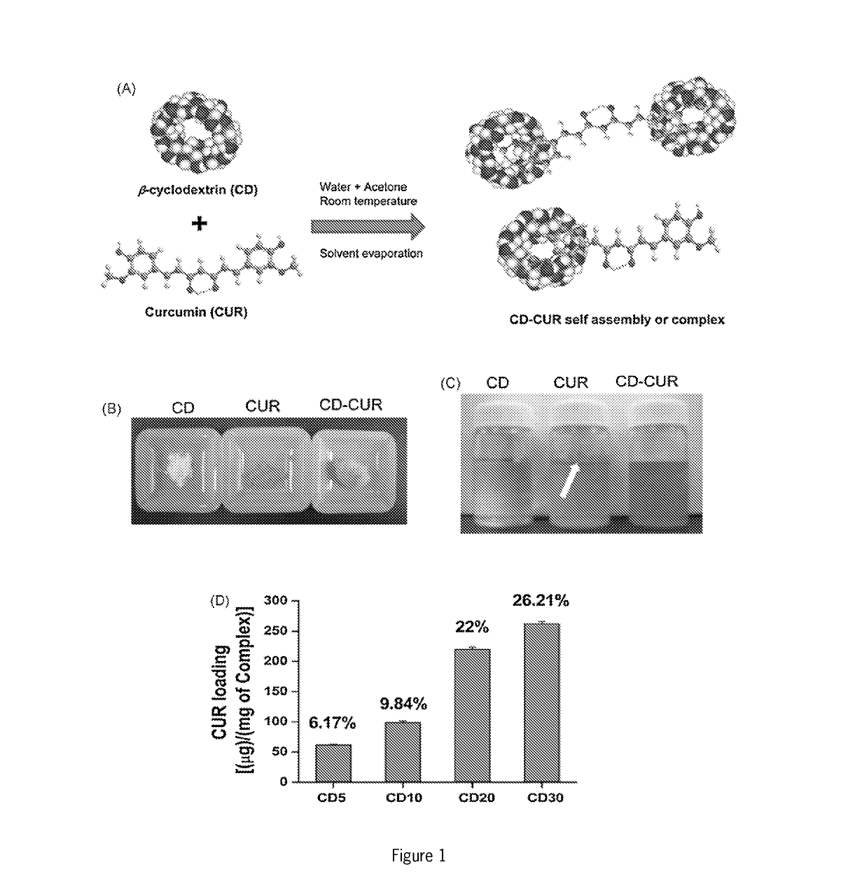

(CD-CUR) self-assembly to enhance the efficacy of curcumin for prostate cancer treatment. It has been reported previously that supramolecular chemistry of β-cyclodextrin offers water soluble inclusion complexes with different drug molecules including anti-cancer chemotherapeutic agents [44-49]. Until now, only a few studies have dealt with increasing the stability and solubility characteristics of CUR to improve bioavailability through CD-CUR inclusion complexes [50-53]. Additionally, no report has focused on the anti-cancer properties of this formulation in prostate cancer cells. In this study, we have explored a supramolecular chemistry approach to stabilize/solubilize curcumin via solvent evaporation encapsulation technique (FIG. 1A). This approach leads to generation of stable curcumin in cyclodextrin cavities (light yellow color fluffy powder) from the CUR (dark yellow powder) and CD (white powder) parent compounds (FIG. 1B). During this process crystalline CD and CUR turned out to be highly amorphous in nature. The yields of CD-CUR inclusion complexes were 34.7 (CD5), 36.4 (CD10), 38.5 (CD20) and 38.2 (CD30) mg.

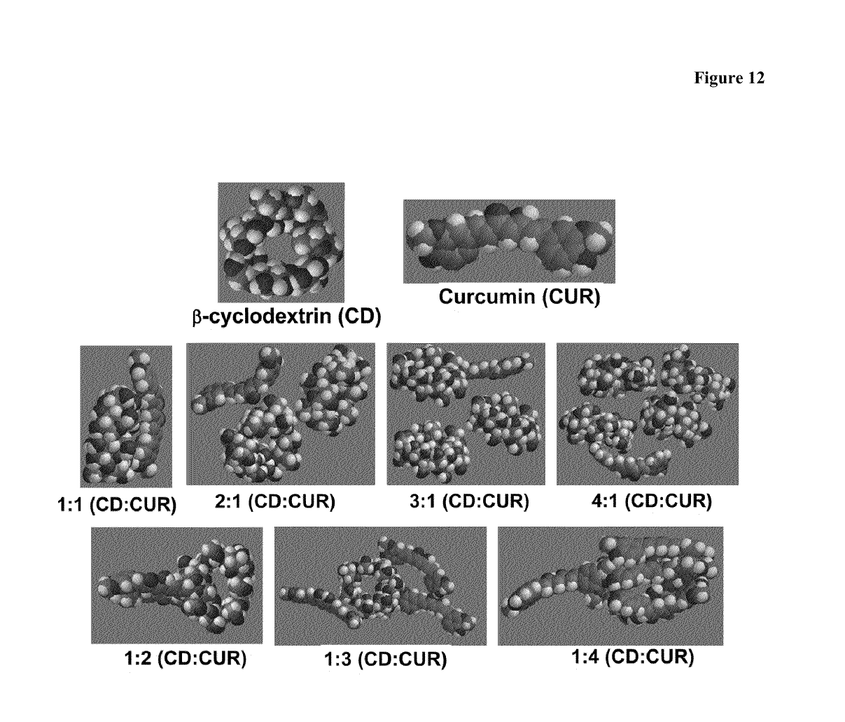

This technique allows us to load or encapsulate different amounts of curcumin into β-cyclodextrin cavities. The feasibility of CUR loading into CD cavities was verified by computer molecular modeling using Sporton'08 Demo Software (Wavefunctions, Inc., Irvine, Calif., USA). This data represents different views of space filling energy minimized molecular models of the three-dimensional conformation of the CD-CUR inclusion complex formation. The host (CD) and guest (CUR) molecules independently build up and their overall possible inclusion complex models were subjected to energy minimization. A few energy minimized molecular model were finalized and presented in FIG. 12.

The loading capacity of CUR in CD cavities increased from CD5 to CD30 self-assemblies (FIG. 1D). The self-assembly process normally results in higher entrapment compared to drug encapsulation into polymer nanoparticles (PLGA nanoparticles). In the case of PLGA nanoparticles, only 5-10% of loading is possible with respect to weight of nanoparticles [32, 33, 53]. This lower drug loading capacity is only suitable for first line chemotherapeutic agents (doxorubicin, Paclitaxel (Taxol®), cisplatin, decitabine, etc.) because even nanomolar concentrations of these drugs act efficiently to kill cancer cells. On the other hand, second line natural therapeutic agents (including curcumin) require a relatively higher dose to kill cancer cells; therefore, a large amount of nanoparticle carriers is required where the nanoparticles also result in some toxicity to the cells. Thus, the current self-assembly process appears to be suitable to load higher amounts of CUR than conventional nanoparticle formulations. According to our study up to 26% of CUR can be encapsulated into the β-CD inclusion complexes (FIG. 1D).

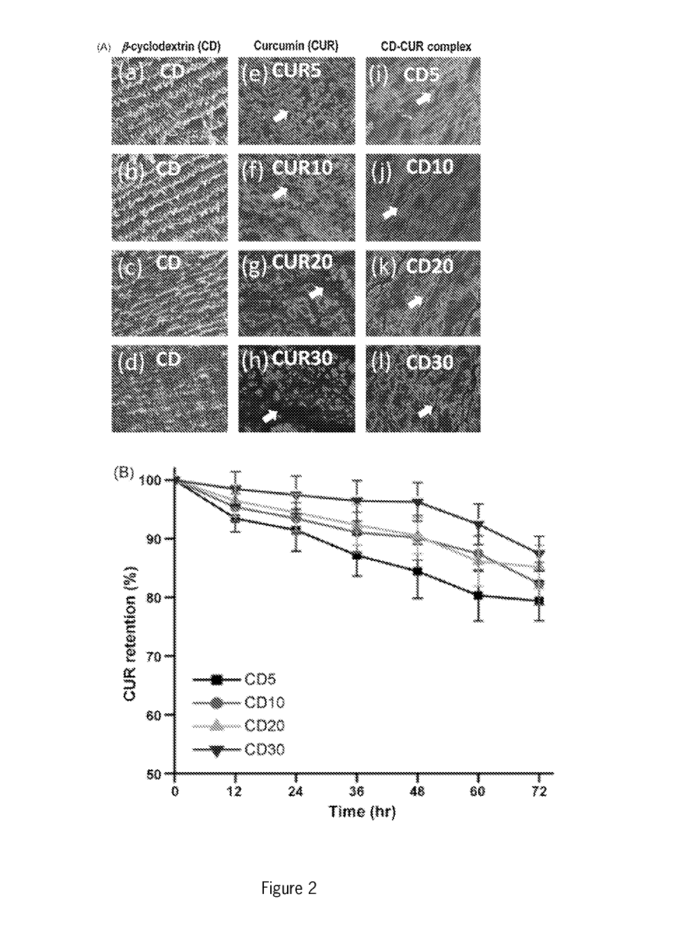

The CD-CUR self-assemblies have shown improvement in the aqueous solubility of curcumin (FIG. 1C, CD-CUR). The pure CUR solubility in PBS solution is ˜20 μg/mL and precipitates in aqueous solutions (FIG. 1C, CUR). It has been determined that the solubility of CD-CUR inclusion complexes are 1.48, 1.62, 1.76 and 1.84 mg of curcumin equivalent CD5, CD10, CD20 and CD30, respectively. A previous study on β-CD-curcumin complex in water reports up to 0.6 mg/mL of CUR solubility [52]. The reason for higher solubility of our CD-CUR formulations in aqueous media is due to better compatibility between CD and CUR in solvent evaporation technique. This phenomenon is confirmed by a compatibility study. CD aqueous dispersion has shown a uniform crystalline rod structures throughout the films (FIG. 2A (a-d)). Since pure curcumin is hydrophobic in nature its aqueous dispersions are highly aggregated and often bulk clumps can be observed (FIG. 2A (e-h) white arrows). Further, the formation of clumps is increased with an increase of CUR concentration in aqueous medium. Whereas, the same concentrations of CUR in CD-CUR inclusion complexes (CD5, CD10, CD20, CD30) aqueous solutions have represented uniform curcumin dispersion along with CD aligned structures (FIG. 2A (i-1)) and no curcumin aggregates/clusters were observed. Therefore, the compatibility study revealed that CD and CUR compounds were dispersed in CD-CUR complexes and showed good compatibility.

The higher compatibility of CD and CUR in CD-CUR complexes may provide an increased in vitro stability of CUR in aqueous medium. All these CD-CUR inclusion complexes have shown high in vitro stability at physiological pH conditions (7.4) (FIG. 2B), whereas CUR did not show significant stability [30,34,51]. The order of stability for 72 hrs was noticed as CD30 (˜87%)>CD20 (˜85%)≧CD10 (˜82.5%)>CD5 (˜79.6%). Only 13% of CUR was precipitated in the case of CD30 while CUR precipitated almost 100%.

### 3.1. Characterization of CD-CUR Inclusion Complexes

### 3.1.1. Spectral Studies

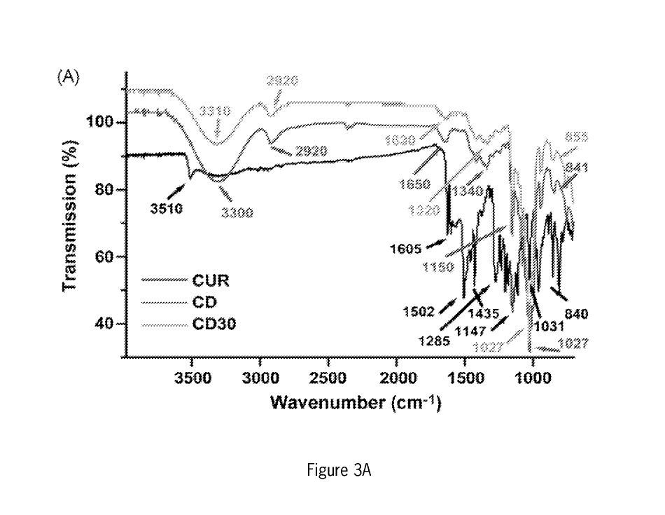

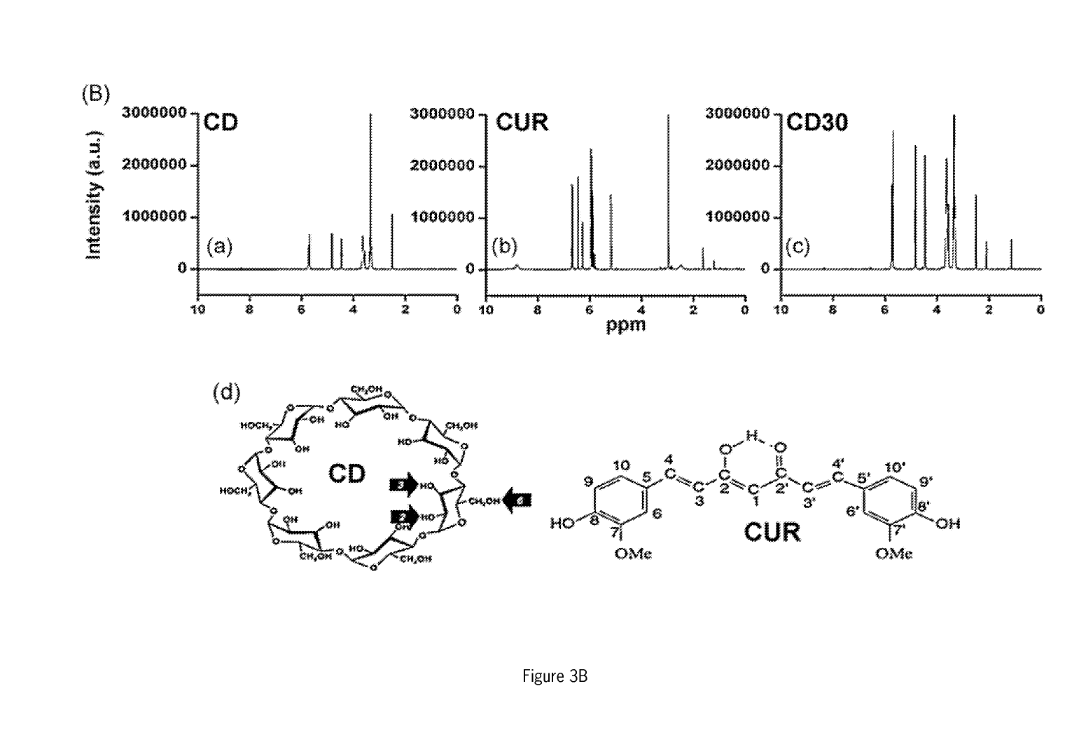

FTIR spectroscopy was used to ascertain the formation of the CD-CUR inclusion complex. The solid samples CD, CUR and CD-CUR inclusion complex (CD30) were recorded for FTIR. The FTIR spectra of CD (black line), CUR (red line) and CD-CUR inclusion complex (CD30) (green line) are represented in FIG. 3A. The FTIR spectrum of curcumin (black line spectrum) exhibited an absorption band at 3510 cm⁻¹ attributed to the phenolic O—H stretching vibration. Additionally, sharp absorption bands at 1605 cm⁻ (stretching vibrations of benzene ring of CUR), 1502 cm⁻¹ (C═O and C═C vibrations of CUR), 1435 cm⁻¹ (olefinic C—H bending vibration), 1285 cm⁻¹ (aromatic C—O stretching vibrations), and 1027/840 cm⁻¹ (C—O—C stretching vibrations of CUR) were noticed. FTIR of CD (red line spectrum) has showed characteristic peaks at 3300 cm⁻¹ and 2920 cm⁻¹ due to the O—H and C—H stretching vibrations. In addition, peaks at 1650 cm⁻¹, 1150 cm⁻¹, 1027 cm⁻¹, and 841 cm⁻¹ correspond to HOH, C—O, C—O—C glucose units and C—O—C of rings of CD, respectively. In the case of CD-CUR inclusion complex spectrum (CD30, green line spectrum), all the sharp peaks belonging to CD have appeared and only few characteristic peaks of CUR are visible. Because of CUR complexation with CD, all the CD related peaks were shifted to higher/lower wave numbers, i.e., 3300 cm⁻¹ to 3310 cm⁻¹; 1650 cm⁻¹ to 1630 cm⁻¹; 1340 cm⁻¹ to 1320 cm⁻¹; 841 cm⁻¹ to 855 cm⁻¹. This data confirmed the presence of CUR in CD-CUR complexes.

This complexation pattern was further evaluated by ¹H-NMR spectroscopy (FIG. 3B). Cyclodextrin exhibited proton signals at δ 5.71 (br s, 2-OH and 3-OH of cyclodextrin), δ 4.83 (br d, H1 of cyclodextrin); δ 4.43 (br s, 6-OH of cyclodextrin), and δ 3.29-3.65 (br m, H2-H6 protons) (FIG. 3B (a)) [43]. Curcumin has showed proton signals at δ 8.2 (8,8′-OH, s 2H), δ 6.71-7.70 (3,3′,4,4′,5,5′,6,6′,7,7′ d, J coupling, total 6H), δ 6.89 (9,9′ d, J, 2H), δ 5.98 (s, 1H) and δ 3.00 (O-Me s, 6H) (FIG. 3B (b)) [54]. CD-CUR inclusion complex (CD30) has shown all the peaks belonging to CD but due to inclusion of CUR their proton signal peaks were shifted to high filed region, i.e., lower δ ppm values (FIG. 3B (c)) [43,54]. However, specific peaks of CUR between 8 and 6 ppm are not visible, which is a typical pattern of an inclusion complex. This data indicate the successful formation of CD-CUR inclusion complexes.

### 3.1.2. Microscopy Studies

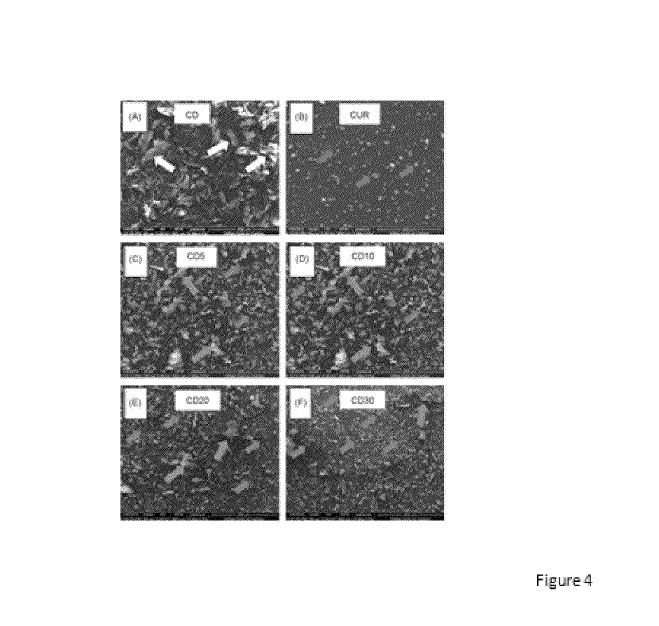

The overall bulk surface morphology of CD, CUR and CD-CUR inclusion complexes (CD5, CD10, CD20, CD30) (for powder) was studied by SEM (FIG. 4). CD shows crystalline flake-like structures throughout the sample (FIG. 4A, white arrows). CUR exhibited highly crystal-like spherical structures (FIG. 4B, red arrows). In the case of CD-CUR inclusion complexes morphology was neither a flake-like structure nor spherical crystals but exhibited as a combination of large (green arrows) and small (blue) aggregates/clumps of CD-CUR assemblies (FIG. 4C-F). This apparent morphological change from flake-like structures to smaller aggregates must be due to curcumin encapsulation in the cavities of CD in CD-CUR complexes. Further, most of the large aggregates disappeared with an increase in encapsulation of CUR in CD cavities, i.e., CD5 to CD30. The CD30 inclusion complex showed a uniform distribution of all CUR molecules inclusion into CD cavities and thus demonstrated very small clusters/aggregates (FIG. 4F) compared to other inclusion complexes (FIG. 4C-E). This alteration of crystal and powder structures further suggests the formation of CD-CUR inclusion complexes.

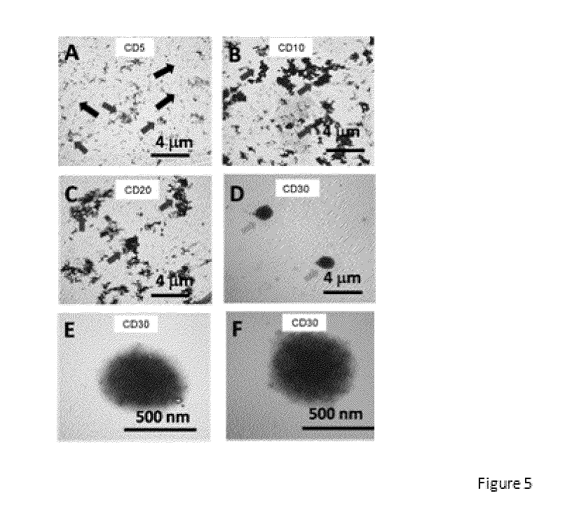

To determine the assembly process of CD-CUR formulation in aqueous medium, a TEM analysis was performed. The TEM analysis of CD-CUR complex samples in solution provides the exact assembly process in aqueous medium (FIG. 5). The CD5 formulation showed less than 100 nm regular assemblies (black arrows) with a few larger clusters (red arrows) (FIG. 5A). The regular assemblies formation is due to few CD-CUR complexes interaction while larger cluster assemblies formation is caused by many CD-CUR complexes. This cluster formation was increased with an increase of CUR in CD inclusion complexation (CD10 and CD 20) (FIG. 5B-C). Because of higher amounts of CUR in CD30 inclusion complex, their self-assemblies resulted in the formation of nanoparticles/nano-assembly of ˜500 nm (FIG. 5D). Magnified nanoparticles self-assemblies shown in FIG. 5E-F clearly indicates self-assembly behavior of CD and CUR inclusion complexes with an apparent size of 500 nm.

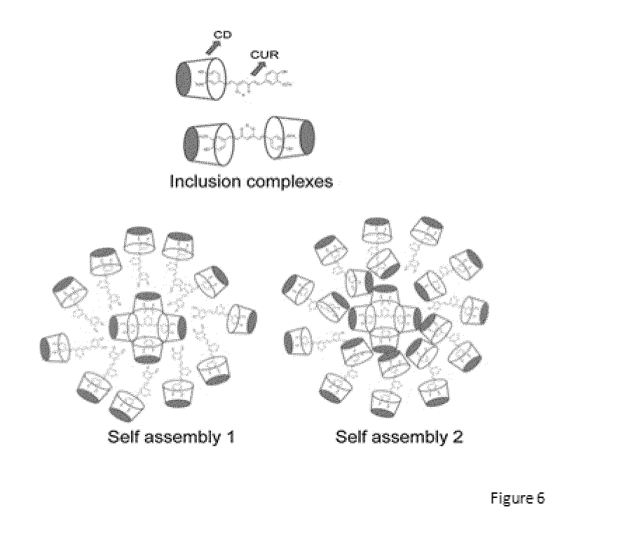

This TEM study also suggests that the inclusion complexation or self-assembly or nanoparticles assembly process between β-cyclodextrin and curcumin occurs via van der Walls interaction, hydrogen bonding and hydrophobic interactions. During the inclusion complex process, high energy water molecules release from the cavity of CD and curcumin molecules enter into it. The feasibility of this entire process was reported for various binary complexes of curcumin with the hydrophobic molecule of CD [52]. Possible mechanisms are proposed for CD-CUR inclusion complex or self-assembly/nano-assembly formation in FIG. 6.

### 3.1.3. Physical State of Complexes

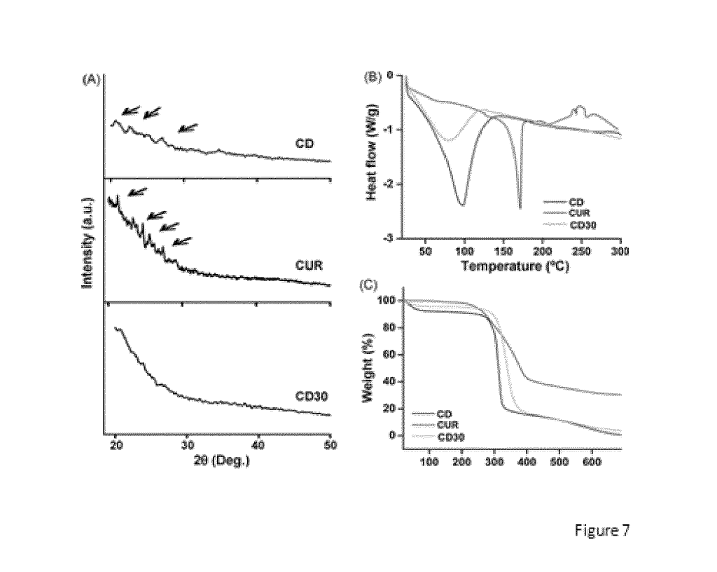

X-ray diffraction (XRD) and thermal methods (DSC and TGA) are useful tools in identifying the physical state of drugs that exist in various polymers, complexes and nanoparticles. In particular, complexation of a drug and cyclodextrin or polymer results in the absence/shifting of endothermic peaks, indicating a change in the crystal lattice, melting, boiling, or sublimation points. Therefore, these measurements can provide both qualitative and quantitative information of drug present in the inclusion complexes. X-ray diffraction patterns of CD, CUR and CD-CUR inclusion complexes are presented in FIG. 7A. Curcumin has displayed the characteristic crystalline peaks of 2θ between 20° and 30° (21.26, 23.35, 24.68 and 26.54°). Few crystalline peaks were also noticed for CD. However, CD-CUR inclusion complex (CD30) did not contain any such crystalline peaks, suggesting the formation of inclusion complex with CD and conversion into amorphous form. This is in accordance with previous reports of CD inclusion complexes [33, 52].

Additionally, the DSC thermograms of CD (black line), CUR (red line) and CD-CUR inclusion complex (CD30) (green line) are shown in FIG. 7B. CD and CUR have shown individual endothermic peaks at 99.5° C. and 172° C., respectively, due to their melting points. Whereas, in the case of CD-CUR inclusion complex, prominent peaks belonging to CUR at 172° C. completely disappeared and also lowered the CD melting point peak to 86.5° C. compared to 99.5° C. This can be attributed to the drug molecules being completely included into the CD cavity by replacing water molecules. Further, this behavior is an indication of stronger interactions between CD and CUR in solid state. Moreover, thermo-gravimetric curves of CD (black line), CUR (red line) and CD-CUR inclusion complex (CD30) (green line) shown in FIG. 7C, demonstrated a complete degradation of CD at 700° C., i.e., weight loss is 100%, while at 700° C., curcumin is degraded only 68%. Unlike CD, CD-CUR formulation (CD30) showed an improved thermal stability due to the presence of CUR (FIG. 7C). This is probably due to formation of CUR inclusion complexation with CD.

Our CD-CUR characterization studies (section 3.1.1. to 3.1.4) clearly demonstrate the formation of CD-CUR inclusion complexes by various self-assemblies depending on the composition. It was also observed in a previous study that complex formation of curcumin and cyclodextrin is highly favored by Gibbs' free energy law [52] which states that CD solutions offer a favorable microenvironment over water for curcumin. The lyophilic cavity of CD protects the lipophilic guest molecule (in this case curcumin) from the aqueous environment, while the polar outer surface of the CD molecules provides the stabilization effect. It has been confirmed that the polarity of the CD cavity is equivalent to 40% solution of ethanol in water [55]. This particular characteristic prompted the use of CD in various pharmaceutical preparations. Here our main aim was to employ the self-assemblies of CD-CUR inclusion complexes as anti-cancer agents.

### 3.2. Cellular Uptake

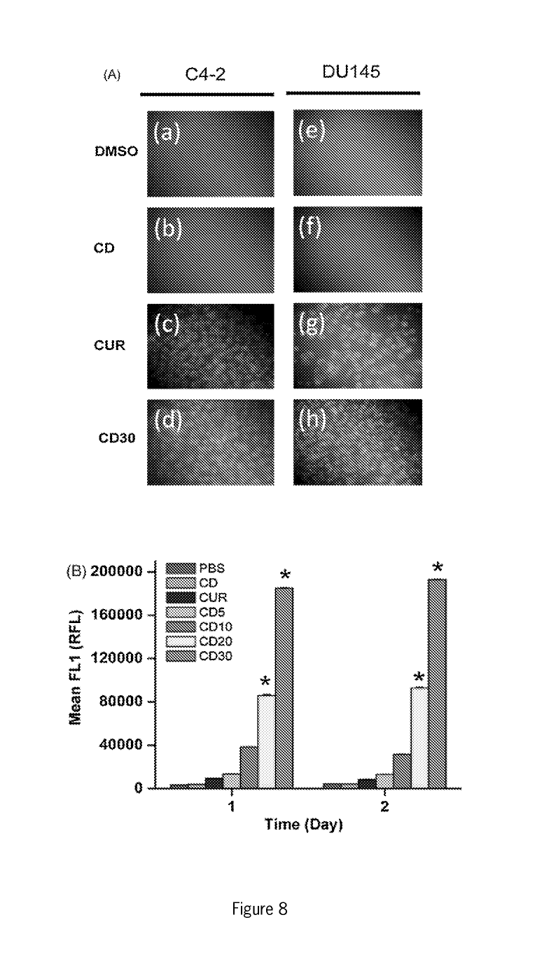

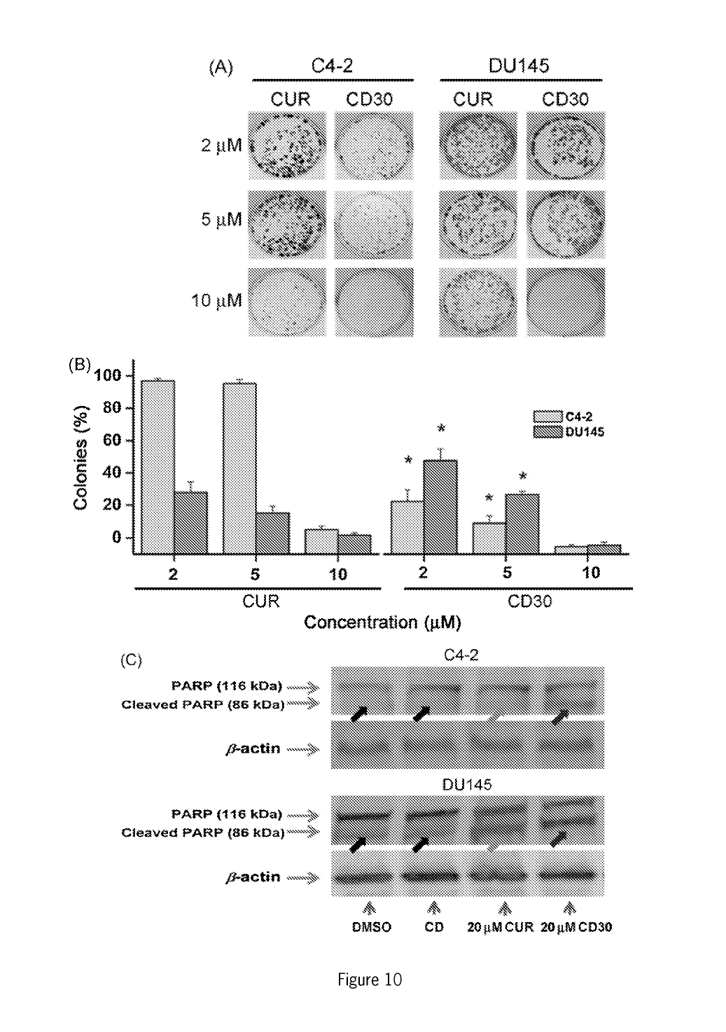

For passive targeting of cancerous tissues, drug loaded cyclodextrins should be able to retain the drug for a prolonged time and capable to enter in cancer cells [56-58]. Curcumin has inherent green fluorescence property which we have utilized in the cellular uptake studies. The microscope images of control cells without curcumin (FIG. 8A, a and e) and cells incubated with cyclodextrin (CD) (FIG. 8A, b and f) did not show any fluorescence in the cells. Whereas, the prostate cancer cells treated with CD-CUR inclusion complex (CD30) formulation exhibited relatively more green fluorescence (FIG. 8A, d and h) compared to cells treated with free curcumin (FIG. 10A, c and g). This data suggest that CD-CUR inclusion complex (CD30) enhanced curcumin uptake in the prostate cancer cells.

Cellular uptake of CUR and all CD-CUR inclusion complexes was investigated by flow cytometry (FACS) to determine which particular complex has a better endocytosis in prostate cancer cells. To determine an exact internalization capability of various CD-CUR inclusion complexes (CD5, CD10, CD20 and CD30), FACS analysis was performed. Free curcumin (CUR) was also used for comparative uptake. For this study, DU145 cells were incubated with 10 μg CUR/CD-CUR formulations for 1 or 2 days. The relative fluorescence values demonstrate a significant increase in intracellular drug uptake of CD-CUR inclusion complexes (CD5, CD10, CD20 and CD30) by cancer cells compared to free CUR (FIG. 8B). The cellular uptake was progressively improved from CD5 to CD30 inclusion complex. However, we did not observe an uptake difference between day 1 and day 2, indicating a rapid uptake process of these inclusion complexes.

Based on this data, it can be speculated that CD30 will exhibit enhanced cytotoxicity compared to CUR and other CD-CUR inclusion complexes (CD5, CD10 and CD20) due to its higher uptake in the prostate cancer cells. Therefore, the anti-cancer efficacy of CD-30 formulation was evaluated in vitro cytotoxicity assays (MTS assay and clonogenic assay) in prostate cancer cell lines.

### 3.3. Anti-Cancer Efficacy of CD30 in Prostate Cancer Cells

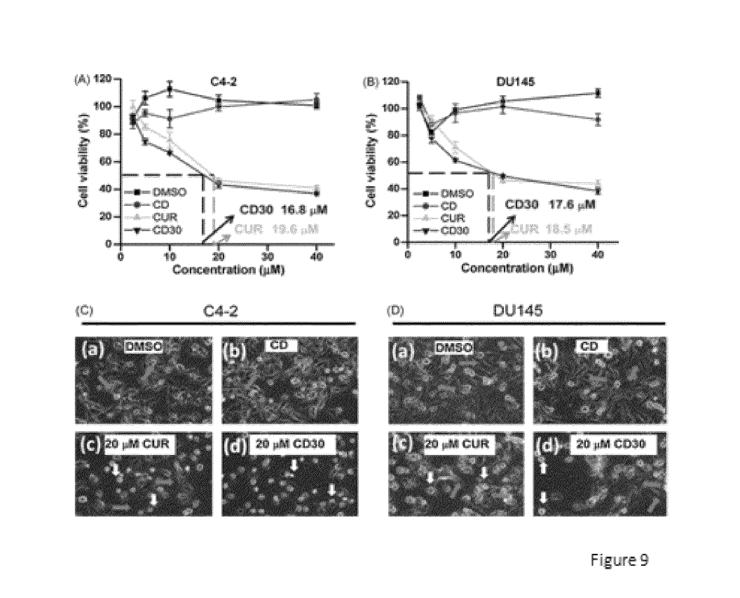

To evaluate anti-cancer efficacy of the CUR and CD30 (CD-CUR inclusion complex), cell proliferation assay was performed in two prostate cancer cell lines [C4-2 (metastatic cell line) and DU145 (non-metastatic cell line)]. These cells were incubated with equivalent doses of 2.5-40 μM CUR or CD30 for 2 days and then examined by MTS assay for cell proliferation. Cells treated with equivalent amounts of DMSO or CD were used as controls for CUR and CD30, respectively. C4-2 and DU145 cancer cells treated with vehicle control, DMSO (FIGS. 9A and B, black lines) or CD (FIGS. 9A and B, red lines) did not show any effect on cell viability which indicates no cytotoxicity of CD or DMSO alone in these cells at indicated concentrations. Whereas, both CUR (FIGS. 9A and B, green lines) and CD30 (FIGS. 9A and B, blue lines) have shown a dose dependent anti-proliferative effect in both C4-2 and DU145 prostate cancer cells. It was noticed that CD30 formulation has a relatively greater anti-proliferative effect compared to free CUR which is evident from their IC₅₀ (concentration of drug required to kill 50% of cells) values (FIGS. 9A and B). The IC₅₀ of CD30 is 16.8 and 17.6 μM in C4-2 and DU145 cancer cells, respectively. Free CUR showed a higher IC₅₀ (C4-2, 19.6 and DU145, 18.4 μM) compared to CD30 in these prostate cancer cells.

The comparative anti-cancer effects of CD30 and CUR is also evident in phase contrast microscopy analysis (FIGS. 9C and D). Similar to MTS assays, C4-2 and DU145 cells treated with vehicle control, DMSO (FIG. 9C a, and FIG. 9D a) or CD (FIG. 9C b, and FIG. 9D b) did not show any cell death and exhibited a greater number of healthy cells (red arrows) in microscopic field. However, the cells treated with 20 μM CUR (FIG. 9C c, and FIG. 9D c) demonstrated the presence of fewer healthy cells (red arrows) and a greater number of dead cells (red arrows) in a microscopic field. Moreover, cells treated with 20 μM CD30 (FIG. 9C d and FIG. 9D d) showed very few healthy cells (red arrows) and most of the dead cells (rounded cells with compromised cell membranes) or cells going towards apoptosis (red arrows). Overall, these data suggest that CUR treatment resulted in a moderate decrease in cell number while CD30 not only reduced cell number but also imparted morphological change related to apoptosis.

To evaluate long-term anti-cancer efficacy of CUR and CD30, colony forming assays (clonogenic assays) were performed in C4-2 and DU145 prostate cancer cell lines. These cells were either treated with equivalent doses (2-10 μM) of CUR or CD30. In colony formation assays, we noticed that cells treated with 2-10 μM CD30 formed fewer colonies when grown over a 10 day period compared to equivalent amount of free CUR treated cells (FIG. 10A). For quantitative analysis, the density of colonies with CD30 or CUR treatments is shown in FIG. 10B. For an example, 2 μM CUR treatments on C4-2 and DU145 cells showed 96% and 74% colonies while with 2 μM CD30 treatment resulted in a drastic decrease in number of colonies (32% and 54% colonies) grown over a 10 day period. Therefore, similar to cell proliferation studies, CD30 treatment showed an improved therapeutic efficacy compared to free curcumin in colony formation assays in prostate cell lines.

In general, therapeutic efficacy of any chemotherapeutic drug can be achieved by its efficient delivery into the cytoplasm. This results only when the drug is released in its active form in cancer cells. We expect that due to self-assembly of CD and CUR, CUR is accumulated, retained, and released in a sustained manner in the cancer cells which causes pronounced effects over free CUR. If that is the case, CD30 must exhibit its superior effects on cellular and molecular pathways. Therefore, we have investigated the expression of Poly(ADP-ribose polymerase (PARP) which is a protein involved in a number of cellular processes, mainly DNA repair and programmed cell death. Cleaved PARP is an indicator of programmed cell death or apoptosis. In response to anti-cancer drug treatment PARP cleavage usually occurs in cancer cells. To determine if our CD30 formulation can effectively induce apoptosis in prostate cancer cells, cells (C4-2 and DU145) were treated with 20 μM CUR or 20 μM CD30 and equivalent amounts of vehicle control (DMSO or CD). The effects of these treatments can be seen in our molecular pathways studies (FIG. 10C). Our immunoblot analysis demonstrated that, CD30 treatment induced cleavage of full length PARP (116 kDa) into cleaved PARP (86 kDa), which allowed cancer cells to undergo cell death or apoptosis (FIG. 10C, blue arrows). The PARP cleavage was more intense in CD30 treatment compared to free CUR treatments (FIG. 10C, green arrows). The control vehicle treatments showed significantly less or no cleavage of full length PARP bands (FIG. 10C, black arrows). These results further suggest an improved efficacy of CD30 formulation on molecular events compared to free CUR in prostate cancer cells. These data also suggest that CD30 self-assemblies may also overcome membrane associated efflux transporter protein and drug resistance [59]. Overall, these data suggest an enhanced efficacy of CD30 formulation compared to free curcumin. Future studies, however, are warranted to investigate the efficacy of this formulation in pre-clinical and clinical models.

### 4. Conclusions

In this study, we have demonstrated feasibility of β-cyclodextrin and curcumin self-assembly via inclusion complexation by using a solvent evaporation technique. Our study showed that curcumin was efficiently encapsulated in β-cyclodextrin cavities and formed different types of self-assemblies. The inclusion complex formation was confirmed by spectral, thermal, X-ray diffraction and electron microscopy studies. CD-CUR inclusion complex (CD30) showed an improved uptake in DU145 cancer cells compared to free CUR. Additionally, this formulation demonstrated greater potent therapeutic efficacy in prostate cancer cells versus free CUR. Our studies suggest that CD30 formulation can be an effective curcumin formulation for prostate cancer therapy.

### REFERENCES

[1] M. Garcia, A. Jemal, E. M. Ward, M. M. Center, Y. Hao, R. L. Siegel and M. J. Thun, Global Cancer Facts & Figures 2007, American Cancer Society, Atlanta, Ga. (2007) 1-46.
[2] A. Jemal, R. Siegel, E. Ward, Y. Hao, J. Xu and M. J. Thun, CA Cancer J. Clin. 59 (2009) 225.
[3] M. Kohli and D. J. Tindall, Mayo Clin. Proc. 85 (2010) 77.
[4] R. Autorino, G. Di Lorenzo, R. Damiano, S. De Placido and M. D'Armiento, Urol. Int. 70 (2003) 1.
[5] W. R. Berry, Urology, 65 (2005) 2.
[6] E. D. Crawford, Urology, 54 (1999) 51.
[7] W. K. Oh, M. H. Tay and J. Huang, Cancer, 109 (2007) 477.
[8] D. P. Petrylak, Urology, 65 (2005) 3.
[9] S. Urakami, H. Shiina, M. Sumura, S. Honda, K. Wake, T. Hiraoka, S. Inoue, N. Ishikawa and M. Igawa, Int. Urol. Nephrol. 40 (2008) 365.
[10] G. Bar-Sela, R. Epelbaum and M. Schaffer, Curr. Med. Chem. 17 (2010) 190.
[11] J. S. Jurenka, Ahern. Med. Rev. 14 (2009) 141.
[12] R. K. Maheshwari, A. K. Singh, J. Gaddipati and R. C. Srimal, Life Sci. 78 (2006) 2081.
[13] A. S. Strimpakos and R. A. Sharma, Antioxid. Redox Signal, 10 (2008) 511.
[14] R. A. Sharma, W. P. Steward and A. J. Gescher, Adv. Exp. Med. Biol. 595 (2007) 453.
[15] B. K. Jaggi, S. C. Chauhan and M. Jaggi, Proceedings of the South Dakota Academy of Science, 86 (2007) 283.
[16] S. Karmakar, N. L. Banik, S. J. Patel and S. K. Ray, Neurosci. Lett. 407 (2006) 53.
[17] S. Shishodia, H. M. Amin, R. Lai and B. B. Aggarwal, Biochem. Pharmacol. 70 (2005) 700.
[18] G. Sa and T. Das, Cell Div. 3 (2008) 14.
[19] P. Anand, C. Sundaram, S. Jhurani, A. B. Kunnumakkara and B. B. Aggarwal, Cancer Lett. 267 (2008) 133.
[20] T. Dorai and B. B. Aggarwal, Cancer Lett. 215 (2004) 129.
[21] M. Hasmeda and G. M. Polya, Phytochemistry, 42 (1996) 599.
[22] J. K. Lin, Arch. Pharm. Res. 27 (2004) 683.
[23] J. Epstein, G. Docena, T. T. Macdonald and I. R. Sanderson, Br. J. Nutr. 103 (2010) 824.
[24] T. Dorai, N. Gehani and A. Katz, Mol. Urol. 4 (2000) 1.
[25] R. L. Hong, W. H. Spohn and M. C. Hung, Clin. Cancer Res. 5 (1999) 1884.
[26] B. B. Aggarwal, A. Kumar and A. C. Bharti, Anticancer Res. 23 (2003) 363.
[27] A. C. Bharti, N. Donato, S. Singh and B. B. Aggarwal, Blood, 101 (2003) 1053.
[28] H. Hatcher, R. Planalp, J. Cho, F. M. Torti and S. V. Torti, Cell. Mol. Life. Sci. 65 (2008) 1631.
[29] R. G. Tunstall, R. A. Sharma, S. Perkins, S. Sale, R. Singh, P. B. Farmer, W. P. Steward and A. J. Gescher, Eur. J. Cancer, 42 (2006) 415.
[30] Y. J. Wang, M. H. Pan, A. L. Cheng, L. I. Lin, Y. S. Ho, C. Y. Hsieh and J. K. Lin, J. Pharm. Biomed. Anal. 15 (1997) 1867.
[31] P. Anand, A. B. Kunnumakkara, R. A. Newman and B. B. Aggarwal, Mol. Pharm. 4 (2007) 807.
[32] S. Bisht, G. Feldmann, S. Soni, R. Ravi, C. Karikar, A. Maitra and A. Maitra, J. Nanobiotech. 5 (2007) 3.
[33] J. Shaikh, D. D. Ankola, V. Beniwal, D. Singh and M. N. Kumar, Eur. J. Pharm. Sci. 37 (2009) 223.
[34] H. H. Tonnesen, Pharmazie, 57 (2002) 820.
[35] Z. Wang, M. H. Leung, T. W. Kee and D. S. English, Langmuir, (2010) DOI: 10.1021/la903772e.
[36] M. H. Leung and T. W. Kee, Langmuir, 25 (2009) 5773.
[37] H. I. Ingolfsson, R. E. Koeppe, 2nd and O. S. Andersen, Biochemistry, 46 (2007) 10384.
[38] K. Maiti, K. Mukherjee, A. Gantait, B. P. Saha and P. K. Mukherjee, Int. J. Pharm. 330 (2007) 155.
[39] A. Safavy, K. P. Raisch, S. Mantena, L. L. Sanford, S. W. Sham, N. R. Krishna and J. A. Bonner, J. Med. Chem. 50 (2007) 6284.
[40] K. T. Al-Jamal, C. Ramaswamy and A. T. Florence, Adv. Drug Deliv. Rev. 57 (2005) 2238.
[41] O. M. Koo, I. Rubinstein and H. Onyuksel, Nanomedicine, 1 (2005) 193.
[42] J. Szejtli, Chem. Rev. 98 (1998) 1743.
[43] G. Horvath, T. Premkumar, A. Bortas, E. Lee, S. Jon and K. E. Geckeler, Mol. Pharm. 5 (2008) 358.
[44] T. Cserhati, E. Forgacs and J. Hollo, J. Pharm. Biomed. Anal. 13 (1995) 533.
[45] P. Y. Grosse, F. Bressolle, P. Rouanet, J. M. Joulia and F. Pinguet, Int. J. Pharm. 180 (1999) 215.
[46] M. Jumaa, B. Carlson, L. Chimilio, S. Silchenko and V. J. Stella, J. Pharm. Sci. 93 (2004) 2953.
[47] N. J. Medlicott, K. A. Foster, K. L. Audus, S. Gupta and V. J. Stella, J. Pharm. Sci. 87 (1998) 1138.
[48] F. Quaglia, L. Ostacolo, A. Mazzaglia, V. Villari, D. Zaccaria and M. T. Sciortino, Biomater. 30 (2009) 374.
[49] J. J. Torres-Labandeira, P. Davignon and J. Pitha, J. Pharm. Sci. 80 (1991) 384.
[50] E. M. Bruzell, E. Morisbak and H. H. Tonnesen, Photochem. Photobiol. Sci. 4 (2005) 523.
[51] H. H. Tonnesen, M. Masson and T. Loftsson, Int. J. Pharm. 244 (2002) 127.
[52] V. R. Yadav, S. Suresh, K. Devi and S. Yadav, AAPS PharmSciTech, 10 (2009) 752.
[53] C. Daniela, G. Marina, T. Michele, E. C. Maria, C. G. Emanuela and C. Giancarlo, J. Incl. Phenom. Macrocycl. Chem. 65 (2009) 391.
[54] M. B. A. Gloria, L. Peret-Almeida, R. J. Alves, L. Dufosse and A. P. F. Cherubino, Food Res. Int. 38 (2005) 1039.
[55] K. H. Fromming and J. Szejtli, Cyclodextrins in pharmacy, Kluwer Academic, Dordrecht, 1994.
[56] B. McCormack and G. Gregoriadis, J. Drug Target, 2 (1994) 449.
[57] P. Caliceti, S. Salmaso, A. Semenzato, T. Carofiglio, R. Fornasier, M. Fermeglia, M. Ferrone and S. Pricl, Bioconjug. Chem. 14 (2003) 899.
[58] E. Bilensoy and A. A. Hincal, Expert Opin. Drug. Deliv. 6 (2009) 1161.
[59] H. R. Mellor and R. Callaghan, Pharmacology, 81 (2008) 275.

We claim:

1. A method for preparing a curcumin formulation, comprising
(a) combining cyclodextrin dissolved in an aqueous solvent with curcumin, or a pharmaceutically acceptable salt, ester, amide, or prodrug thereof, in a non-aqueous solvent, wherein the combining occurs with agitation and under conditions to allow evaporation of the non-aqueous solvent; and
(b) separating solid and supernatant phases of the combination; wherein the supernatant phase contains cyclodextrin-curcumin inclusion complexes.

2. The method of claim 1, wherein the non-aqueous solvent is selected from the group consisting of dimethyl sulphoxide, dimethyl formamide, chloroform, dichloromethane, dioxane, ethanol, methanol, and acetone.

3. The method of claim 1, wherein the cyclodextrin is selected from the group consisting of β-cyclodextrin, α-cyclodextrin, γ-cyclodextrin, and modifications thereof.

4. The method of claim 1, wherein the aqueous solvent is water.

5. The method of claim 1, wherein the separating comprises centrifugation.

6. The method of claim 1, wherein the combining comprises stirring at 200-600 rpm, and wherein the combination is stirred in a container with no cap.

7. The method of claim 6, wherein the stirring is carried out for between 1-24 hours.

8. The method of any claim 1, wherein the separating step comprises centrifugation at between 800-1200 rpm.

9. The method of claim 1, wherein the non-aqueous solvent is acetone and the aqueous solvent is water.

10. The method of claim 1, wherein the cyclodextrin is dissolved in the aqueous solvent at a concentration of between 4-12 mg/ml.

11. The method of any claim 1, wherein the cyclodextrin is dissolved in the aqueous solvent at a concentration of between 6-10 mg/ml.

12. The method of claim 1, wherein the cyclodextrin is dissolved in the aqueous solvent at a concentration of about 8 mg/ml.

13. The method of claim 1, wherein the curcumin, or pharmaceutically acceptable salt, ester, amide, or prodrug thereof, is dissolved in the non-aqueous solvent at a concentration of between 5-15 mg/ml.

14. The method of claim 1, wherein the curcumin, or pharmaceutically acceptable salt, ester, amide, or prodrug thereof, is dissolved in the non-aqueous solvent at a concentration of between 7.5-12.5 mg/ml.

15. The method of claim 1, wherein the curcumin, or pharmaceutically acceptable salt, ester, amide, or prodrug thereof, is dissolved in the non-aqueous solvent at a concentration of about 10 mg/ml.

16. The method of claim 1, wherein the method further comprises isolation of β-cyclodextrin-curcumin inclusion complexes from the supernatant.

17. The method of claim 16, wherein the isolation comprises freeze-drying the supernatant.

18. The method of claim 1, wherein the combining comprises combining β-cyclodextrin and curcumin, or pharmaceutically acceptable salt, ester, amide, or prodrug thereof, in a ratio of between 20:1 and 1:1.

19. The method of claim 1, wherein the combining is initially carried out at room temperature.

20. A cyclodextrin-curcumin inclusion complex.

21. The cyclodextrin-curcumin inclusion complex of claim 20, wherein the cyclodextrin and curcumin are present in a ratio of between 20:1 and 3.33:1.

22. The cyclodextrin-curcumin inclusion complex of claim 20, wherein the cyclodextrin comprises β-cyclodextrin.

23. A self-assembly comprising a plurality of the cyclodextrin-curcumin inclusion complexes of claim 20.

24. A pharmaceutical dosage form comprising the cyclodextrin-curcumin self-assembly of claim 23.

25. The pharmaceutical dosage form of claim 24, wherein the dosage form is formulated into form selected from the group consisting of tablets, gelcaps, softgels, and capsules.

26. An isolated cyclodextrin-curcumin inclusion complex, or self-assembly thereof, prepared by the method of claim 1

27. A method for treating one or more disorder selected from the group consisting of inflammation, infection, stroke, and cancer, comprising administering to a subject in need thereof a cyclodextrin-curcumin self-assembly thereof, according to claim 24 in an amount effective to treat the inflammation, infection, or cancer.
